(19) United States
(12) Patent Application Publication
(10) Pub. No.: US 2007/0092898 A1
(43) Pub. Date: Apr. 26, 2007

(54) Detection of multiple anti-viral antibodies

(75) Inventor: Huaizhong Hu, Madison, WI (US)

Correspondence Address: Medlen & Carroll, LLP, Suite 350, 101 Howard Street, San Francisco, CA, 94105, US

(73) Assignee: Renovar Incorporated, Madison, WI (US)

(21) Appl. No.: 11/504,510

(22) Filed: Aug. 15, 2006

Related U.S. Application Data
(60) Provisional application No. 60/708,209, filed on Aug. 15, 2005.

(51) Int. Cl. C12Q 1/68; C12N 5/06; C12N 5/16

(52) U.S. Cl. 435/6; 435/345

(57) ABSTRACT

The present invention relates to methods and kits for the diagnosis and monitoring of hepatitis C virus (HCV) infection and/or Kaposi's sarcoma herpesvirus infection (KSHV) in a subject, as well as other infectious diseases and agents. In particular, the present invention relates to the diagnosis and monitoring of infectious disease through the use of multiple agents directed at a target of interest. For example, the present invention provides for the detection of HCV infection by the detection of antibodies to HCV C22 core, NS 4 (C-10003), NS3 and NS5 antigens, and/or to the diagnosis and monitoring of KSHV infection by detection of antibodies to K8.1, orf73/LNA1 and orf65 antigens in serum. The present invention further relates to methods and kits for assessing the efficacy of agents and interventions used to treat infectious diseases.

The present invention claims priority to U.S. Provisional Application Ser. No. 60/708,209, filed Aug. 15, 2005, the disclosure of which is herein incorporated by reference in its entirety.

The present invention was funded in part under U.S. Public Health Service grant No. N43-CP-31148. The government may have certain rights in the invention.

### FIELD OF THE INVENTION

The present invention provides methods and kits for diagnosing, detecting, and monitoring a wide variety of infectious diseases. For example, the present invention provides kits and methods for analysis of blood and other bodily fluids for the presence or absence of infectious disease agents. For example, the present invention relates to methods and kits for the diagnosis and monitoring of hepatitis C virus (HCV) infection and/or Kaposi's sarcoma herpes virus infection (KSHV) in a subject, as well as other infectious diseases and agents. In particular, the present invention relates to the diagnosis and monitoring of infectious disease through the use of multiple agents directed at a target of interest. For example, the present invention provides for the detection of HCV infection by the detection of antibodies to HCV C22 core, NS 4 (C-10003), NS3 and NS5 antigens, and/or to the diagnosis and monitoring of KSHV infection by detection of antibodies to K8.1, orf73/LNA1 and orf65 antigens in serum. The present invention further relates to methods and kits for assessing the efficacy of agents and interventions used to treat infectious diseases.

### BACKGROUND OF THE INVENTION

Battling infectious diseases remains a pressing problem facing the medical community. For example, infections of hepatitis C virus (HCV) and/or Kaposi's sarcoma-associated herpes virus (KSHV) are major health problems in the world. Hepatitis C is a blood borne virus previously referred to as non-A/non-B hepatitis. It was not until 1992 that a reliable blood test was developed to identify the antibody against the hepatitis C virus (HCV) (1). HCV enters the body through direct blood exposure and attacks and kills liver cells where it multiplies. This process causes inflammation in the liver and results in the death of liver cells. The incubation period varies from 2 to 26 weeks. Many people report little or no initial symptoms during the acute phase. However, mild flu-like symptoms including nausea, fatigue, fever, headaches, loss of appetite and abdominal pain can occur. A minority of individuals report severe flu-like symptoms with jaundice and/or dark urine. It is believed that as many as 85% of people initially infected with HCV become chronically infected, if the person does not clear the infection within a 6-month period (2). The disease will then progress over a period of 10-40 years with some individuals sustaining liver damage that will lead to cirrhosis and/or liver cancer and may require liver transplantation. However, many people do not have symptoms and are leading relatively normal lives. 4.5 million people are infected with HCV in the U.S. and 300 million worldwide. Of those infected in the U.S only half are aware that they are infected. 85% of infections become chronic, and if left untreated there is 20-30% chance of developing cirrhosis, liver cancer or liver failure. There are 230,000 new infections in the U.S. every year from HCV and that number is expected to triple by the year 2010 (1, 3).

Kaposi's sarcoma (KS) is a rare tumor observed especially in allograft patients and/or patients with AIDS. It was first identified in 1872, when only sporadic cases were found world-wide (4). In recent years with the advent of AIDS, Kaposi's sarcoma has emerged as a frequent diagnostic consideration for a new population of patients, predominantly homosexual men, who are at risk of a more aggressive form of KS (4-7). Kaposi's sarcoma-associated herpes virus (KSHV), also known as human herpesvirus-8 plays a critical role in the development of KS. HHV-8 DNA sequences have been detected in 100% of amplifiable samples from AIDS patients with KS, and 15% of non-KS tissue samples from AIDS patients. The virus can be isolated from PBMC as well as KS tumor cells. To date, a number of studies with KS tissues from AIDS patients have identified the presence of HHV-8. Currently, KS is the most common cancer in AIDS patients, affecting approximately 20% of people with HIV-1 infection (4-7). Human herpesvirus-8 has been identified in almost 95% of KS tumors. HHV-8 has subsequently been found to be associated with multicentric Castleman's disease and primary effusion lymphoma. There have also been reports of HHV-8's association with multiple myeloma (MM). Interest in human herpesvirus-8 is also of growing significance in solid organ transplantation (4-7).

Detection of serum antibodies directed at viral antibodies is a standard method to indicate viral infection. In terms of detecting human antibody response to HCV and/or KSHV, ELISA is sensitive and well automated, but each ELISA antibody must be measured separately if the titer of that antibody must be known. Furthermore, detection of antibodies directed at different viruses must presently be conducted separately (8-15). Commercially available kits presently used for HCV diagnosis are for the most part based on ELISA. First generation ELISA kits relied on a fusion protein antigen produced from an original 5-1-1 clone with a few adjoining clones. The antigen was denoted as the C100 antigen. The problem with the first generation kits was that the antigen used was non-structural and thus may not pick up all cases of HCV. Furthermore, antibodies against this antigen could not be detected until 15 weeks after the onset of hepatitis. Therefore, HCV infection cannot be excluded in those whose serum is antibody-negative up to 6 months after the onset of symptoms (7-10). The first generation kits had also been demonstrated to have a poor specificity. Second and third generation assays are now used for serological diagnosis. Second generation assays incorporate C22 core antigen as well as NS4 (C-100-3) and NS3 antigens. The newer third generation assays incorporate NS5 as an additional antigen. However this increase in sensitivity is offset by a decrease in specificity. This has reduced the “diagnostic window” down to 4 weeks after initial infection.

Clearly, a method is needed that can simultaneously detect multiple antibodies in a single test. Such an assay would have many advantages over traditional ELISA-based methods including greater efficiency, less need for operator intervention, and conservation of scarce serum samples.

### SUMMARY OF THE INVENTION

The present invention provides methods and kits for diagnosing, detecting, and monitoring a wide variety of infections diseases. For example, the present invention provides kits and methods for analysis of blood and other bodily fluids for the presence or absence of infectious disease agents. For example, the present invention relates to methods and kits for the diagnosis and monitoring of hepatitis C virus (HCV) infection and/or Kaposi's sarcoma herpes virus infection (KSHV) in a subject, as well as other infectious diseases and agents. In particular, the present invention relates to the diagnosis and monitoring of infectious disease through the use of multiple agents directed at a target of interest. For example, the present invention provides for the detection of HCV infection by the detection of antibodies to HCV C22 core, NS 4 (C-10003), NS3 and NS5 antigens, and/or to the diagnosis and monitoring of KSHV infection by detection of antibodies to K8.1, orf73/LNA1 and orf65 antigens in serum. The present invention further relates to methods and kits for assessing the efficacy of agents and interventions used to treat infectious diseases. The present invention is not limited to HCV or KSHV detection. In some embodiments, other infectious diseases and agents include, for example, antigens and/or antibodies selected from the group comprising hepatitis B core antibody (anti-HBc), hepatitis C virus antibody (anti-HCV), HIV-1 and HIV-2 antibodies (anti-HIV-1 and anti-HIV-2), HTLV-I and HTLV-II antibodies (anti-HTLV-I and anti-HTLV-II), hepatitis B surface antigen (HbsAg) and syphilis. In other embodiments, other infections diseases and agents include, for example, antigens and antibodies directed to antigens specific for pathogens described in, for example, Fields Virology (B. N. Fields, editor, Lippincott, Williams and Wilkins, 2001), and/or Principles and Practice of Infectious Diseases (G. L. Mandel et al., editors, Churchill Livingstone, 2004), each of which is herein incorporated by reference in their entireties.

In a preferred embodiment, a multiplex assay is provided that detects a multiplicity of infectious disease agents simultaneously from a single sample of a pool or mixture of samples. In one such embodiment, a Luminex system is used to detect a plurality of infectious disease agents, wherein beads are associated with one or more antibodies that specifically detect each target agent. Such an embodiment provides an easy to use, single assay for screening all relevant viral antigens, antibodies, or other agents in a sample of interest. This assay is particularly useful for blood screening, including the screening of pooled samples of blood (e.g., pooled from two or more individuals).

Accordingly, in some embodiments, the present invention provides a method for the detection of antibodies directed separately at HCV and/or KSHV antigens in a single assay on a single sample from a single subject.

In one embodiment, the present invention provides a method of diagnosing Kaposi's sarcoma herpes virus (KSHV) infection, comprising providing a sample from a subject, wherein said subject is suspected of having KSHV infection, and reagents for particle-based flow cytometric detection of one or more antibodies from the group comprising antibodies to KSHV K8.1, orf73, and orf65 antigens, and detecting the presence of said one or more antibodies in said sample using said reagents. In some embodiments, two or more antibodies directed at different regions of a KSHV protein or different proteins are used (e.g., in a particle-based flow cytometric system) in a reaction mixture to detect the presence KSHV with a high sensitivity.

In a further embodiment, the present invention provides a kit for use in the above methods. For example, the kit may comprise one or more of: a) reagents for particle-based flow cytometric detection of at least one antibody from the group comprising antibodies to KSHV K8.1, orf73, and orf65 antigens; b) instructions for using said reagents for detecting the presence of at least one of said antibodies; and c) instructions for using said detection of at least one of said antibodies in said sample for diagnosing KSHV infection.

In another embodiment, the present invention provides a method of diagnosing hepatitis C virus (HCV) infection comprising providing a sample from a subject, wherein said subject is suspected of having HCV infection, and reagents for particle-based flow cytometric detection of at least three or more antibodies from the group comprising antibodies to HCV core, NS3, NS4, and NS5 antigens, and detecting the presence of said antibodies in said sample using said reagents. In some embodiments, two or more antibodies directed at different regions of a HCV protein or different proteins are used (e.g., in a particle-based flow cytometric system) in a reaction mixture to detect the presence HCV with a high sensitivity.

In still another embodiment, the present invention provides a kit comprising reagents for particle-based flow cytometric detection of at least three or more antibodies from the group comprising antibodies to HCV core, NS3, NS4, and NS5 antigens instructions for using said reagents for detecting the presence of one or more of said antibodies, and instructions for using said detection of said antibodies in said sample for diagnosing HCV infection.

In a preferred embodiment, the present invention provides a method for the diagnosis of HCV and/or KSHV infection in a subject comprising providing a sample from a subject, wherein said subject is suspected of having an infection caused by at least one virus selected from the group comprising HCV and/or KSHCV and reagents for particle-based flow cytometric detection of at least three antibodies from the group comprising antibodies to KSHV K8.1, orf73, and orf65 antigens, and/or to HCV core, NS3, NS4, and NS5 antigens, and detecting the presence of said antibodies in said sample using said reagents.

In a particularly preferred embodiment, the present invention provides a kit comprising reagents for particle-based flow cytometric detection of at least three antibodies from the group comprising antibodies to KSHV K8.1, orf73, and orf65 antigens, and/or to HCV core, NS3, NS4, and NS5 antigens, instructions for using said reagents for detecting the presence of one or more of said antibodies, and instructions for using said detection of said antibodies in said sample for diagnosing HCV and/or KSHV infection. In some embodiments, said instructions comprise instructions required by the United States Food and Drug Administration for use in in vitro diagnostic products.

The present invention additionally provides a method of determining a treatment course of action, comprising providing a sample from a subject, wherein the subject is suspected of having HCV or KSHV infection and detecting the amount of the HCV and/or KSHV antibodies in the sample using the reagents, and determining a treatment course of action based on the detecting. In some embodiments, the treatment course of action comprises continued monitoring. In some embodiments, the present invention further comprises the step of determining a treatment course of action based on the prediction of HCV or KSHV infection. In some embodiments, the treatment course of action comprises the administration of therapeutic agents. In some embodiments, the treatment course of action comprises a surgical procedure. In additional embodiments the surgical procedure comprises solid organ transplantation.

The present invention also provides a method of screening compounds, comprising providing a sample from a subject, wherein the subject is suspected of having HCV or KSHV infection, an assay with reagents for detection of HCV and/or KSHC antibodies, and one or more test compounds, and administering the test compound to the subject, and detecting the amount of the HCV and/or KSHC antibodies in the sample using the reagents. The present invention is not limited to a particular sample type. Any bodily fluid including, but not limited to, blood, urine, serum, and lymph may be utilized. In some preferred embodiments, the sample is a serum sample. In some embodiments, the test compound is a drug. In some embodiments, the method further comprises the step of determining the efficacy of the drug based on the detecting.

In further embodiments, the present invention provides methods and kits for the diagnosis and monitoring of antibodies in a sample from a subject specific for antigens expressed by viral pathogens, for example, hepatitis B virus, hepatitis A virus, herpes simplex virus, human papilloma virus, human immunodeficiency virus, or other human viral pathogen. In preferred embodiments, the particle-based flow cytometric assays of the present invention provide methods and kits for the simultaneous diagnosis and monitoring of antibodies specific for infection by two or more, or five or more, or ten or more viral pathogens in a single assay in a single sample from a single subject.

### DESCRIPTION OF THE FIGURES

FIG. 1 shows a design of the particle-base flow cytometric assay for detecting antibodies directed at HCV and/or KSHV antigens in some embodiments of the present invention.

FIG. 2 shows the effect of sample dilution on fluorescent signal intensity derived from antibodies directed at HCV core antigen in 8 human sera (except HCV2, all are HCV positive samples).

FIG. 3 shows the effect of sample dilution on fluorescent signal intensity derived from antibodies directed at HCV NS3 antigen in 8 human sera.

FIG. 4 shows the effect of sample dilution on fluorescent signal intensity derived from antibodies directed at HCV NS4 antigen in 8 human sera.

FIG. 5 shows the effect of sample dilution on fluorescent signal intensity derived from antibodies directed at HCV NS5 antigen in 8 human sera.

FIG. 6 shows the effect of sample dilution on fluorescent signal intensity derived from antibodies directed at KSHV K8.1 antigen in 8 human sera.

FIG. 7 shows the effect of sample dilution on fluorescent signal intensity derived from antibodies directed at KSHV orf65 antigen in 8 human sera.

FIG. 8 shows the effect of sample dilution on fluorescent signal intensity derived from antibodies directed at KSHV orf73 antigen in 8 human sera.

### DEFINITIONS

To facilitate an understanding of the present invention, a number of terms and phrases are defined below:

As used herein, the term “fluorescently activated cell sorting assay” (FACS) refers to any assay suitable for use in cell sorting techniques (e.g., flow cytometry) that employs detection of fluorescent signals.

As used herein, the terms “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IgE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject.

As used herein, the term “surgical procedure” refers to any procedure that involves treatment of injury, deformity, or disease by manual or instrumental means.

As used herein, “reagents for detection of at least one compound” and “reagents for detection of two or more compounds” refer to reagents specific for detection of the infectious disease antibodies of the present invention. In some embodiments, the reagent is an antibody. In other embodiments, the reagent is aptamer. In other embodiments, the reagents and kits of the present invention further comprise additional reagents and devices for performing detection assays, including, but not limited to, controls, buffers, and substrates (for example, beads, microspheres, and microarrays).

As used herein, the terms “instructions for using said reagents for detecting the presence of one or more said compounds”, and “instructions for using said detecting the presence of one or more said compounds in said sample for diagnosing HCV or KSHV infection” include instructions for using the reagents contained in the kit for the diagnosis of a viral infection in a sample from a subject. In some embodiments, the instructions further comprise the statement of intended use required by the U.S. Food and Drug Administration (FDA) in labeling in vitro diagnostic products. Information required in an application may include: 1) The in vitro diagnostic product name, including the trade or proprietary name, the common or usual name, and the classification name of the device; 2) The intended use of the product; 3) The establishment registration number, if applicable, of the owner or operator submitting the submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A summary of the safety and effectiveness data upon which the substantial equivalence determination is based; or a statement that the safety and effectiveness information supporting the FDA finding of substantial equivalence will be made available to any person within 30 days of a written request; 7) A statement that the submitter believes, to the best of their knowledge, that all data and information submitted in the pre-market notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

As used herein, the term “determining a treatment course of action” as in “determining a treatment course of action based on said diagnosis of a viral infection” refers to the choice of treatment administered to a patient. For example, if a patient is found to be at increased risk of a HCV or KSHV infection, therapy may be started, increased, or changed from one treatment type (e.g., pharmaceutical agent, surgery) to another. Conversely, if a patient is found to be at low risk for a HCV or KSHV infection, therapy may not be administered or levels of therapy may be decreased. In some embodiments, the treatment course of action is “continued monitoring” in which no treatment is administered but the levels of HCV or KSHV antibodies measured in the patient's sample is monitored regularly (e.g., using the diagnostic methods of the present invention). In other embodiments, the “treatment course of action” as used herein, comprises use of the results of the HCV or KSHV infection assays of the present invention as indicators of the need for additional tests of a HCV or KSHV infection, for example, an imaging scan, biopsy or endoscopically guided exam.

As used herein, the term “determining the efficacy of HCV or KSHV infection drugs based on said detecting” refers to determining if a drug is preventing HCV or KSHV infection based on, for example, detecting the level of HCV or KSHV antibodies in the serum of a patient who manifests signs and symptoms of, or is at risk for HCV or KSHV infection.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence (that is, the “full size” sequence) minus one amino acid.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the terms “protein microarray” and “protein chip” refer to protein-detecting molecules immobilized at high density on a substrate, and probed for various biochemical activities. (See, for example: Zhu H and Snyder M, “Protein chip technology”, Current Opinion in Chemical Biology 7: 55-63, 2003; Cutler P, “Protein arrays: The current state of the art”, Proteomics 3; 3-18, 2003; and MacBeath G, “Protein microarrays and proteomics”, Nature Genetics Supplement 32: 526-532, 2002, each of which is incorporated herein by reference in its entirety).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (for example, HCV and/or KSHV infection). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include urine and blood products, such as plasma, serum and the like. Such examples are not however to be construed as limiting the sample types applicable to the present invention.

### DETAILED DESCRIPTION OF THE INVENTION

The present invention provides methods and kits for diagnosing, detecting, and monitoring a wide variety of infections diseases. For example, the present invention provides kits and methods for analysis of blood and other bodily fluids for the presence or absence of infectious disease agents. For example, the present invention relates to methods and kits for the diagnosis and monitoring of hepatitis C virus (HCV) infection and/or Kaposi's sarcoma herpes virus infection (KSHV) in a subject, as well as other infectious diseases agents. In some embodiments, other infectious diseases and agents include, for example, antigens and/or antibodies selected from the group comprising hepatitis B core antibody (anti-HBc), hepatitis C virus antibody (anti-HCV), HIV-1 and HIV-2 antibodies (anti-HIV-1 and anti-HIV-2), HTLV-I and HTLV-II antibodies (anti-HTLV-I and anti-HTLV-II), hepatitis B surface antigen (HbsAg) and syphilis. In other embodiments, other infections diseases and agents include, for example, antigens and antibodies directed to antigens specific for pathogens described in, for example, Fields Virology (B. N. Fields, editor, Lippincott, Williams and Wilkins, 2001), and/or Principles and Practice of Infectious Diseases (G. L. Mandel et al., editors, Churchill Livingstone, 2004), each of which is herein incorporated by reference in their entireties. In particular, the present invention relates to the diagnosis and monitoring of infectious disease through the use of multiple agents directed at a target of interest. For example, the present invention provides for the detection of HCV infection by the detection of antibodies to HCV C22 core, NS 4 (C-10003), NS3 and NS5 antigens, and/or to the diagnosis and monitoring of KSHV infection by detection of antibodies to K8.1, orf73/LNA1 and orf65 antigens in serum. The present invention further relates to methods and kits for assessing the efficacy of agents and interventions used to treat infectious diseases.

Infections of hepatitis C virus and/or Kaposi's sarcoma-associated herpes virus (KSHV) are major health problems in the world. Detection of serum antibodies directed at viral antibodies is a standard method to indicate viral infection. With regard to detection of the human antibody response to HCV and/or KSHV, the enzyme linked immunosorbent assay (ELISA) is sensitive and may be automated. Nevertheless, each anti-viral antibody assays have to be measured separately if the titer of that antibody is required. Furthermore, detection of antibodies directed at different viruses has to be conducted separately. A method that can simultaneously detect several antibodies in one test has many advantages over traditional ELISA, including efficiency, less operator intervention, and conservation of serum samples. Systems and methods of the present invention (e.g., employing cytometric technology) achieve this goal. It is possible to combine the detection of antibodies directed at one or more infectious disease antigens and/or multiple different proteins or regions of proteins associated with a single antigen or multiple antigens (e.g., HCV, KSHV, etc.) in a single assay or single reaction vessel (e.g., using the particle-based technology of the present invention). This combined assay using the particle-based cytometric assays is several times more efficient than using ELISA. In work conducted in the course of the development of the present invention, simultaneous detection of multiple anti-viral antibodies in human blood in a single assay has been shown to be technically feasible (see Example 2, Tables 1 and 2) and provide superior results. Compared to traditional ELISA-based methods, the particle-based flow cytometric assay of the present invention is capable of far greater efficiency requiring much less serum from each individual. This flow cytometric assay is particularly useful for projects that must process large numbers of samples, for example, an epidemiologic survey or donor blood screening in blood banking. Moreover, the particle-based cytometric assay of the present invention represents a diagnostic method for patients suffering from viral infections including, for example, HCV and/or KSHV infection.

Methods

Detection and quantification of antibodies against certain viral antigens have been shown to be predictive of subsequent risk of cancer or other serious diseases. However, progress in the area has been limited by serologic techniques developed in the last century, many of which require subjective interpretation, and by substantial labor and other costs required for titering to quantify antibody reactivities. In one embodiment of the present invention, it is now possible to overcome many of these limitations. This technique has been modified to replace traditional labor-intensive immunoassays such as enzymatic linked immunosorbant assay (ELISA) or radio-immunoassay (RIA). In one embodiment of the present invention, a particle-based assay, for example, has been developed to simultaneously detect antibodies directed at hepatitis C virus (HCV) and/or Kaposi's sarcoma herpes virus (KSHV). Such an assay finds utility both in research activities and in clinical diagnosis.

The present invention is not limited to a particular detection assay. In some embodiments, antibodies are detected by binding of a capture molecule specific for the antibody (for example, an aptamer, or an antibody). In some embodiments, HCV and/or KSHV antibodies are detected by binding of an antibody specific for the protein (i.e., an immunoassay). The present invention is not limited to a particular capture molecule or antibody. Any capture molecule or antibody (e.g., monoclonal or polyclonal) that detects target antibodies may be utilized. Exemplary detection assays are described herein.

Antibody binding is detected by techniques known in the art. For example, in some embodiments, antibody binding is detected using a suitable technique, including but not limited to, radio-immunoassay, ELISA (enzyme-linked immunosorbant assay), “sandwich” immunoassay, immunoradiometric assay, gel diffusion precipitation reaction, immunodiffusion assay, precipitation reaction, agglutination assay (e.g., gel agglutination assay, hemagglutination assay, etc.), complement fixation assay, immunofluorescence assay, protein A assay, and immunoelectrophoresis assay.

In some preferred embodiments, a quantitative ELISA assay is utilized (See e.g., U.S. Pat. Nos. 5,958,715, and 5,484,707, each of which is herein incorporated by reference). In some preferred embodiments, the quantitative ELISA is a competitive ELISA. In a competitive ELISA, the wells of a microtiter plate are first coated with a fusion protein comprising all or a fragment of the HCV and/or KSHV antibodies. The sample to be tested is added to the plate along with an antibody that is specific for the HCV and/or KSHV antibodies. The HCV and/or KSHV antibodies in the sample compete for binding to the antibody with the immobilized peptide. The plate is washed and the antibody bound to the immobilized HCV and/or KSHV antibodies is then detected using any suitable method (e.g., a secondary antibody comprising a label or a group reactive with an enzymatic detection system). The amount of signal is inversely proportional to the amount of HCV and/or KSHV antibodies present in the sample (e.g., a high signal is indicative of low amounts of HCV and/or KSHV antibodies being present in the urine).

In some embodiments, an automated detection assay is utilized. Methods for the automation of immunoassays include, but are not limited to, those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a diagnosis and/or prognosis based on the level of HCV and/or KSHV antibodies in the sample is utilized. In other embodiments, the immunoassay described in U.S. Pat. Nos. 5,789,261, 5,599,677 and 5,672,480, each of which is herein incorporated by reference, is utilized.

In still other embodiments, a protein microarray or protein chip array assay is utilized for detection (See e.g., U.S. Pat. No. 6,197,599, herein incorporated by reference). In such an assay, proteins (e.g., antibodies specific for HCV and/or KSHV antibodies) are immobilized on a solid support such as a chip. A sample suspected of containing HCV and/or KSHV antibodies is passed over the solid support. Bound HCV and/or KSHV antibodies are then detected using any suitable method. In some embodiments, detection is via surface plasmon resonance (SPR) (See e.g., WO 90/05305, herein incorporated by reference). In SPR, a beam of light from a laser source is directed through a prism onto a biosensor consisting of a transparent substrate, usually glass, which has one external surface covered with a thin film of a noble metal, which in turn is covered with an organic film that interacts strongly with an analyte, such as a biological, biochemical or chemical substance. The organic film contains antibodies (e.g., specific for HCV and/or KSHV antibodies of the present invention), which can bind with an analyte (e.g., HCV and/or KSHV antibodies) in a sample to cause an increased thickness, which shifts the SPR angle. By either monitoring the position of the SPR angle, or the reflectivity at a fixed angle near the SPR angle, the presence or absence of an analyte in the sample can be detected.

In other embodiments, The PROTEINCHIP (Ciphergen Biosystems, Fremont, Calif.) is utilized for detection. The PROTEINCHIP system uses SELDI (Surface-Enhanced Laser Desorption/Ionization) technology to perform the separation, detection and analysis of proteins at the femtomole level directly from biological samples (See e.g., U.S. Pat. No. 6,294,790 and U.S. Patent Application US20010014461A1, each of which is herein incorporated by reference). In the PROTEINCHIP technology, proteins of interest (e.g., HCV and/or KSHV antibodies) are captured on the PROTEINCHIP Array (e.g., via a bound antibody) directly from the original source material. The chip is washed to remove undesired materials and bound proteins are detected using SELDI.

In some embodiments, a cytometric bead array assay is used (Quantum Plex kit, Bangs Laboratories; Cytometric Bead Array kit, BD Biosciences). These systems allow for multiple analyte detection with small volume samples. In other embodiments, a Luminex bead assay is used.

The present invention is not limited to the detection of HCV and/or KSHV antibodies in serum. Any bodily fluid that contains elevated levels of HCV and/or KSHV antibodies correlated with HCV and/or KSHV infection may be utilized, including, but not limited to, blood, serum, urine, lymph, bile, cerebrospinal fluid and saliva.

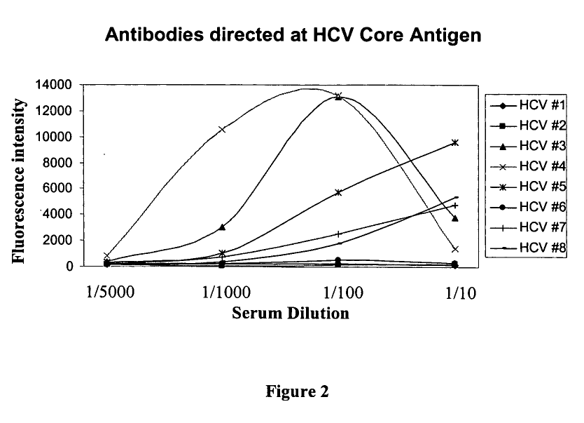

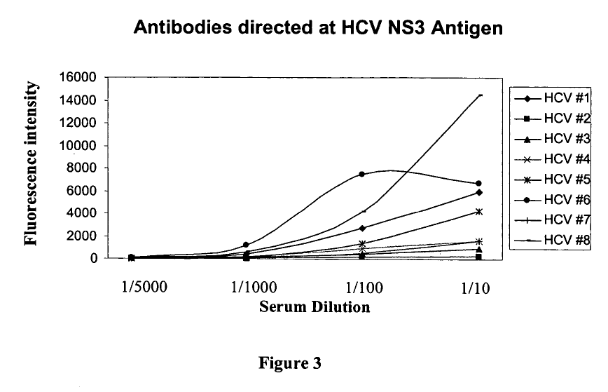

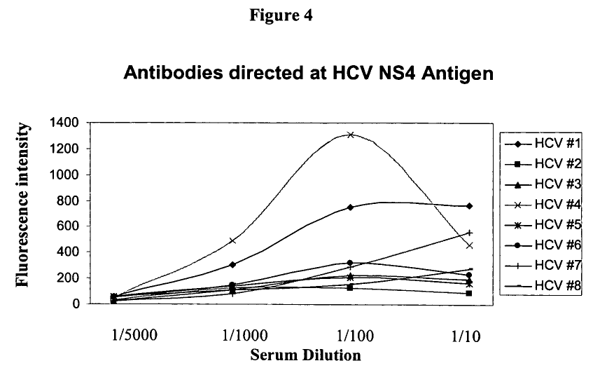

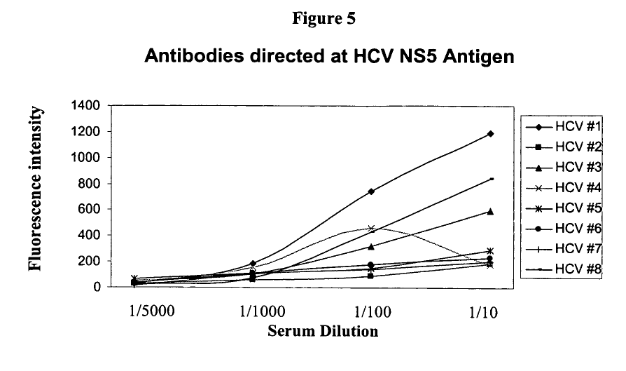

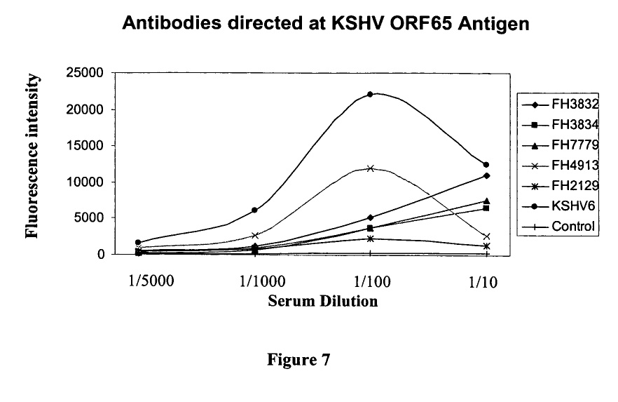

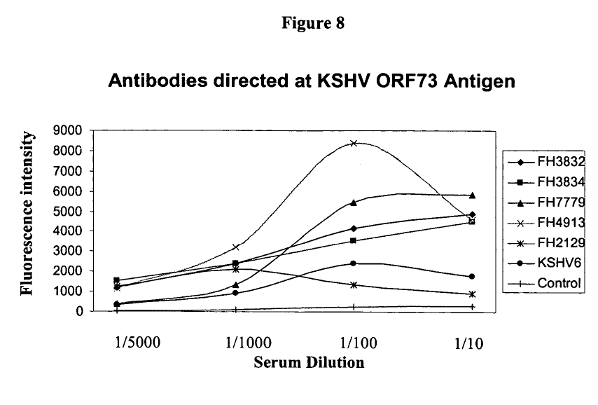

In some particularly preferred embodiments, a combination of multiple HCV and/or KSHV or other infectious disease antibodies or epitopes are detected simultaneously in body fluid samples. In some embodiments, other infectious diseases and agents include, for example, antigens and/or antibodies selected from the group comprising hepatitis B core antibody (anti-HBc), hepatitis C virus antibody (anti-HCV), HIV-1 and HIV-2 antibodies (anti-HIV-1 and anti-HIV-2), HTLV-I and HTLV-II antibodies (anti-HTLV-I anti-HTLV-II), hepatitis B surface antigen (HbsAg) and syphilis. In some embodiments, the method uses fluorescence dye labeled beads that can detect multiple (e.g., at least 3), for example HCV and/or KSHV antibodies, in one assay. In an exemplary embodiment (Example 2), the assay was used to detect multiple HCV and/or KSHV antibodies. Detection of these HCV and/or KSHV antibodies was conducted in the same test tube simultaneously as depicted in Tables 2 and 3. As the HCV and/or KSHV antibody concentration increases, the mean fluorescence intensity for each group of beads increases (FIGS. 2-8). This correlation between the HCV and/or KSHV antibody concentration and the mean fluorescence establishes the basis for this particle-based cytometric quantitative method. A standard curve for each HCV and/or KSHV antibody has been constructed. These results demonstrate a quantitative assay for the simultaneous detection of multiple HCV and/or KSHV antibodies.

In some embodiments, testing is performed in a clinical (e.g., hospital or clinic) setting. In such embodiments, testing is generally ordered and interpreted by a physician or other clinician. In some embodiments, testing is carried out by a lab technician (e.g., in an in-house or external clinical lab). In preferred embodiments, clinical testing utilizes a quantitative assay for detection of antibodies. In some embodiments, testing is utilized to determine the likelihood of organ failure in a patient e.g., liver failure. In other embodiments, testing is utilized to monitor organ function in a subject who has recovered from an infection, and is not on medication. In still further embodiments, testing is utilized to monitor the effectiveness of a medication. In some embodiments, the antibodies test is used to complement other clinical or laboratory investigations, for example biopsy or metabolite level in serum, and to monitor response to therapy. In a preferred embodiment, the antibody tests of the present invention are used as a reference parameter in deciding whether and when a biopsy should be taken.

The HCV and/or KSHV antibody test of the present invention is simple to conduct and rapid, making it suitable for clinical use. Based on the result of the clinical testing, the appropriate intervention is taken e.g., including, but not limited to, an increase or decrease in levels of drug therapy, initiation of drug therapy, termination of therapy, surgery, further testing, or continued monitoring.

In some embodiments, the present invention provides drug-screening assays (e.g., to screen for drugs effective in treating HCV or KSHV infection). The screening methods of the present invention utilize the detection of antigen-specific antibodies. For example, in some embodiments, the present invention provides methods of screening for compounds that alter (e.g., increase or decrease) the expression of antigen-specific antibodies. In some embodiments, the levels of antigen-specific antibodies are detected (e.g., using a method described herein) in a subject that has undergone administration of a candidate compound. The increased levels of antigen-specific antibodies are indicative of a candidate compound that is not preventing infection. Conversely, preferred candidate compounds are those that reduce antigen-specific antibodies.

In some embodiments, drug-screening assays are performed in animals. Any suitable animal may be used including, but not limited to, baboons, rhesus or other monkeys, mice, or rats. Animal models of HCV and/or KSHV infections are generated (e.g., by the administration of HSV and/or KSHV), and the effects of candidate drugs on the animals are measured. In preferred embodiments, HSV and/or KSHV infections in the animals are measured by detecting levels of HCV and/or KSHV antigen-specific antibodies in the serum of the animals. The level of HCV and/or KSHV antigen-specific antibodies may be detected using methods and kits of the present invention.

Kits

Given the value of the particle-based flow cytometric assay embodiments of the present invention for detecting anti-viral antibodies in research and clinical practice, a well-standardized kit is suitable for these purposes. In one embodiment, the present invention provides a particle-based flow cytometric assay kit that detects and quantifies antibodies directed at HCV and/or KSHV or other infectious disease agents singly or simultaneously in combination. This kit consists of a complete or partial set of reagents, computer analysis software compatible with commercially available software packages, and instructions sufficient for detection and quantification of the antibodies. The kit yields reproducible results, and is easy to use thereby surpassing traditional ELISA-based methods in efficiency and precision.

In some embodiments, the present invention provides kits for the detection of HCV and/or KSHV antibodies. In some embodiments, the kits contain antibodies specific for HCV and/or KSHV antibodies in addition to detection reagents, buffers or devices. In some embodiments, the kits contain reagents and/or instructions for testing for concurrent infections. In preferred embodiments, the kits contain all of the components necessary to perform a detection assay, including all controls, directions for performing assays, and any necessary hardware or software for analysis and presentation of results.

In further embodiments, the particle-based cytometric assay kits of the present invention generate a computerized results report. Before acquisition of the data, the combination of bead number and correspondent antigen or antibody is input into the Luminex system. Following that, sample identification and dilution factors are entered. After the data acquisition, the Luminex system automatically reports the results. The reported data can be directly exported, for example, to Microsoft Excel and data management may be conducted in a mode that the users are familiar with. If users wish to have a special format for data analysis, they may create their own analysis software.

In preferred embodiments, the particle-base cytometric assay kits of the present invention contain reagents and detailed protocols used for detecting anti-HCV and/or KSHV antibodies in human samples from, for example, serum. In one embodiment, the detection platform is the Luminex 100 IS analyzer. The user prepares a set of 96 well plate vacuum washing system, and purchases 96 well plates for the antibody detection. Exemplary components of the kits of the present invention may include, for example, antigen coupled beads, biotin labeled goat anti-human IgG antibody/biotin labeled goat anti-human albumin antibody (for positive control), streptavidin-PE, 8 control sera (Optional; the user may prepare their own control sera), dilution buffer, and washing buffer. Exemplary steps of the protocols of the kits of the present invention may include, for example, an introduction to the Luminex multiple parameter assay and the use of the platform to detect anti-HCV and/or KSHV antibodies in human serum, an overview of kit components and storage conditions, an inventory of materials and equipment to be provided by the user, flow charts of the assay steps, instructions for data acquisition by the Luminex analyzer, and directions for data analysis including the recommended calculation equation and thresholds.

Antibodies

The present invention provides isolated antibodies. In preferred embodiments, the present invention provides monoclonal antibodies that specifically bind to antigen-specific antibodies. These antibodies find use in the diagnostic methods described herein. In other embodiments, commercially available antibodies are utilized (e.g., available from any suitable source including, but not limited to, R & D System, Minneapolis, Minn.).

An antibody of the present invention may be any monoclonal or polyclonal antibody, as long as it can recognize the infectious disease-specific antibodies or antigens of interest. Antibodies can be produced by using antibodies or expressed infectious disease proteins in the subject as the antigen according to a conventional antibody or antiserum preparation process. The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., HCV and/or KSHV antigen-specific antibodies). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against HCV and/or KSHV antigen-specific antibodies) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared, and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to a hapten in a weight ratio of about 0.1 parts to about 20 parts, preferably, about 1 part to about 5 parts per 1 part of the hapten.

In addition, various condensing agents can be used for coupling of a hapten and a carrier. For example, glutaraldehyde, carbodiimide, maleimide-activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody. Further, fragments of the antigen-specific antibodies may be used. Fragments may be obtained by any methods including, but not limited to expressing a fragment of the gene, enzymatic processing of the protein, chemical synthesis, and the like.

### EXPERIMENTAL

The following examples are provided in order to demonstrate and further illustrate certain preferred embodiments and aspects of the present invention, and are not to be construed as limiting the scope thereof.

Serum samples and reagents: HCV positive and negative serum samples were purchased from BBI Diagnostics (West Bridgewater, Mass.). KSHV positive serum samples were a kind gift from NCI (Bethesda, Md.). HCV NS4, NS3 and core antigenic peptides were purchased from Biodesign International (Saco, Me.), and HCV NS5 antigenic peptide was purchased from Maine Biotechnology Services (Portland, Me.). KSHV K8.1, orf73, and orf65 antigenic peptides were a kind gift from the NCI (Bethesda, Md.). Luminex beads were purchased from Luminex Corporation (Austin, Tex.). Biotinylated goat anti-human IgG antibody and streptavidin-PE were purchased from BD PharMingen (San Jose, Calif.). Antibodies directed at human albumin are purchased from Research Diagnostics (Flanders, N.J.). Antibodies directed at HCV core/NS3/NS4 regions are purchased from Biodesign International (Saco, Me.). Rat monoclonal antibody directed at KSHV ORF-73 is purchased from Advanced Biotechnologies (Columbia, Md.). 96-well filter plates will be purchased from Millipore (Bedford, Mass.).

Coupling antigens peptides to Luminex beads: Coupling of HCV and KSHV antigenic peptides to Luminex carboxylated microspheres was conducted following the Luminex coupling protocol. Coupled beads were stored at 4° C. and were shown to be stable for at least 3 months. After beads coupling, the goat antibody directed at HCV core/NS3/NS4 and the rat antibody directed at KSHV ORF-73 are used to examine the coupling efficiency.

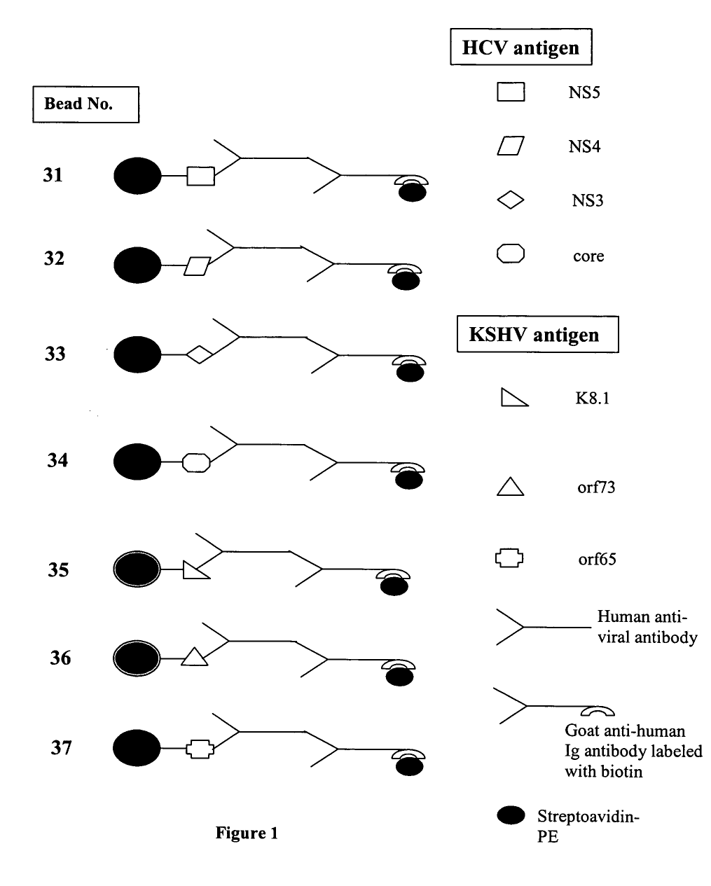

Determination of anti-HCV and/or anti-KSHV antibodies: The design of the experiment is shown in FIG. 1. Twenty five μl of beads with (100 beads/μl of each type) were mixed with 50 μl of appropriately diluted serum sample in a 96-well plate. The reaction was incubated in the dark at room temperature for 60 min. After vacuuming and washing once with 200 μl PBS supplemented with 0.1% BSA, the beads were resuspended in 75 μl PBS/BSA, and 25 μl of biotinylated goat anti-human IgG antibody (1:250 dilution) was added to each well. The reaction was incubated in the dark at room temperature for 60 min. Afterwards 25 μl of streptavidin-PE was added to each well. Data acquisition was performed on a Luminex 100 IS analyzer after a 30 min incubation.

### Example 1

Sample Dilution Tests

Serum dilution is important for antibody detection. In many semi-quantitative tests, antibody titers are reported. The titer reporting methods are labor intensive, because each serum sample needs to be diluted many fold, and each dilution needs to be evaluated. A quantitative method can overcome this drawback, but this test should be conducted at an appropriate serum dilution to achieve the highest sensitivity. A dilution test was therefore to determine the most appropriate dilution for further study.

As shown in FIGS. 2-5, 8 HCV positive/negative serum samples were used in the experiment. Each sample was diluted at 1/10, 1/100, 1/1000, and 1/5000. The experiment was conducted with the 4 HCV antigenic peptide-conjugated beads in the same reaction. While the fluorescence intensity was highest for most samples at a 1/10 dilution, there were 2-3 samples that showed a significant drop in fluorescence intensity from 1/100 to 1/10 dilution. For example, HCV #3 and HCV #4 in reacting to HCV core antigen peptide behaved in this manner.

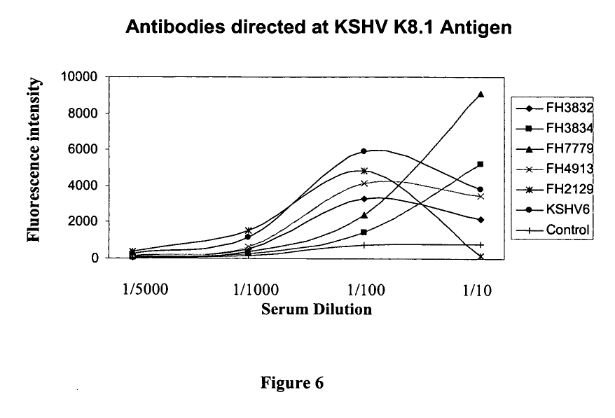

The same dilution test was performed for KSHV positive serum samples as well. Each sample was diluted at 1/10, 1/100, 1/1000, and 1/5000. The experiment was conducted with the 3 KSHV antigenic peptide-conjugated beads in the same reaction. The results are presented in FIGS. 6-8. Similar to the HCV test, the fluorescence intensity reached the highest level for some samples at 1/10, but for other samples at 1/100 dilution.

These results indicate that the detection of multiple antibodies directed at HCV or KSHV can be done in one assay simultaneously with the present Luminex platform. Given that the fluorescence intensity is fairly high at 1/100 dilution for viral antibody-positive samples, the 1/100 is the appropriate dilution for further quantitative experiments.

### Example 2

Simultaneous Detection of Antibodies Directed at HCV and KSHV in One Assay

In order to combine the detection of antibodies directed at both HCV and the KSHV antigens in the same assay, protocols have been developed to detect serum anti-viral antibodies using the particle-based flow cytometric assay. As developed, this method is simple and user-friendly. A representative protocol is as follows:

Exemplary HCV and KSHV Reaction Protocol

1. Mix 25 μl of beads (100 of each type of bd/μl in PBS/TBN) with 50 μl of serum sample at appropriate dilution in a 96-well plate.
2. Incubate in the dark for 1 hr
3. Vacuum and wash reaction wells each with 200 μl of PBS/BSA once
4. Resuspend beads in 75 μl of PBS/BSA for each well
5. Add 25 μl to each well of biotin labeled anti-human IgG at a 1 to 250 dilution
6. Incubate in the dark for 1 hr
7. Add 25 μl to each well of streptavidin phycoerythrin (PE) (1 to 25 dilution)
8. Incubate for 30 minutes
9. Add 25 μl to each well of Stop buffer and mix
10. Read results on Luminex
   a. Use the template HCV 4-plex for the HCV tests
   b. Use the template KSHV 3-plex for KSHV tests
   c. Use the template HCV-KSHV 7-plex for HCV and KSHV multiplexed assays

This protocol requires approximately 3.5 hours to complete one round of data acquisition. It is much simpler than the traditional ELISA that normally takes more than 6 hours, and has many fewer steps of manual work. Nevertheless, it is possible to further reduce the incubation time, for example, from 60 min to 40 min.

To generate serum samples that are known to be positive for both viruses, we mixed serum samples of HCV positive and KSHV positive patients to study the possibility of simultaneously detecting the antibodies directed at both viruses. Ten μl of each serum sample was added to 980 μl of sample buffer, yielding a 1 to 100 dilution of each sample (for example: mix 1=HCV #1+FH3832). The dilution in the mixed samples is therefore the same as the single sample test. Because these serum samples are pooled from two people, the results generated may not be the same as that obtained from the assay of single serum sample, although the general trend should be maintained.

In our previously performed dilution test on serum samples directed at either HCV or KSHV we found that 1:100 was the most suitable dilution for the antibody detection using the present Luminex Detection Platform. Therefore, all serum samples in the current experiment were diluted at 1:100 for detecting the antibodies. Furthermore, although the fluorescence unit (or intensity) reported for each sample by the Luminex Detector is quantitative, it may be variable from one experiment to another for the same sample. This is due to the variation of the reagents and incubation time. Therefore, reporting the fluorescence unit will not be suitable for comparison between experiments. To solve this problem, we used control serum samples to set up a baseline. After being divided by the average fluorescence intensity of these control samples, a relative score can be obtained for each antibody of each serum sample. The score is a relative number based on the internal control using the control serum samples, and it should be much more consistent than the fluorescence intensity of each sample from one experiment to another.

Following these principles, we performed experiments using the serum samples to simultaneously detect antibodies directed at both HCV and KSHV. Table 1 shows the original fluorescence intensity, and Table 2 presents the normalized scores of antibodies directed at each viral antigen. Table 1 shows fluorescence intensity of serum antibodies directed at HCV and KSHV antigens. The experiment was conducted simultaneously with beads coated with HCV and KSHV antigens mixed together. Mix 1=HCV1+FH3832, Mix 2=HCV2+FH3834, Mix 3=HCV3+FI2129, Mix 4=HCV4+FF4913, Mix 5=HCV5+FG7779, Mix 6=HCV6+KSHV6. #: HCV2 is a negative sample according to BBI Diagnostics from which we purchased the samples.

TABLE 1

| | | | | | KSHV | KSHV | KSHV |
|---|---|---|---|---|---|---|---|
| Sample | HCV core | HCV NS3 | HCV NS4 | HCV NS5 | K8.1 | ORF65 | ORF73 |
| HCV1 | 86.5 | 1965 | 581 | 420 | 113 | 140.5 | 30 |
| HCV2^{#} | 58.5 | 138.5 | 71 | 63.5 | 96.5 | 98 | 11.5 |
| HCV3 | 11510 | 283.5 | 154 | 137 | 50 | 297 | 95 |
| HCV4 | 18011 | 711 | 655 | 220.5 | 141.5 | 322.5 | 108 |
| HCV5 | 4200 | 1095.5 | 126 | 132 | 57.5 | 552 | 32 |
| HCV6 | 331 | 7159 | 250.5 | 164 | 168 | 193.5 | 53.5 |
| HCV7 | 2255 | 452.5 | 171 | 107 | 101 | 163.5 | 53.5 |
| HCV8 | 1285 | 3483 | 52 | 230.5 | 84.5 | 106.5 | 22 |
| HCV9 | 152 | 7299 | 97 | 40 | 33.5 | 421.5 | 40 |
| HCV10 | 3764.5 | 4189 | 350 | 281 | 430 | 145 | 50.5 |
| FH3832 | 292.5 | 79.5 | 76.5 | 134.5 | 3120 | 3255.5 | 4323.5 |
| FH3834 | 265.5 | 85 | 104 | 85 | 1440 | 2814 | 3912.5 |
| FI2129 | 300 | 78 | 100 | 103 | 4960 | 2144 | 3293 |
| FF4913 | 501.5 | 97 | 145 | 121 | 2970 | 5749 | 7457 |
| FG7779 | 298 | 77 | 143.5 | 78.5 | 1694 | 1810 | 4379 |
| KSHV6 | 406 | 179 | 262 | 161.5 | 4819 | 17078 | 2319.5 |
| Mix1 | 266 | 1368.5 | 332 | 356 | 2831 | 3171.5 | 3136 |
| Mix1 | 298 | 1735 | 453 | 419.5 | 3286 | 3098.5 | 4464.5 |
| Mix2 | 292 | 176 | 113.5 | 85 | 946 | 2232 | 2967 |
| Mix2 | 269 | 142.5 | 138.5 | 130 | 1178 | 2467.5 | 3564 |
| Mix3 | 10338.5 | 250 | 139.5 | 174 | 5322 | 2566 | 3352.5 |
| Mix3 | 9893 | 301.5 | 145 | 201 | 5670 | 2667.5 | 3422 |
| Mix4 | 14774 | 518 | 371 | 182.5 | 2431.5 | 5643 | 6580 |
| Mix4 | 12910 | 474 | 332 | 127 | 2258.5 | 5074.5 | 5543 |
| Mix5 | 3173.5 | 618 | 128 | 72 | 1627 | 2050 | 2857 |
| Mix5 | 2772.5 | 620 | 114 | 94 | 1282 | 1770 | 2287.5 |
| Mix6 | 543 | 5026 | 339.5 | 262.5 | 4424 | 17728.5 | 2276.5 |
| Mix6 | 571 | 5997 | 365 | 223 | 4754 | 19182.5 | 2263 |
| control 1 | 85 | 39 | 71 | 40.5 | 28.5 | 136 | 48 |
| control 2 | 208 | 231.5 | 71.5 | 84.5 | 291 | 176 | 117 |
| control 3 | 206 | 138 | 86 | 46 | 144 | 92 | 52 |
| control 4 | 169.5 | 86.5 | 24 | 31 | 151 | 114 | 13 |
| control 5 | 129 | 13 | 21 | 30.5 | 19 | 94 | 1.5 |
| control 6 | 97.5 | 72 | 17 | 37 | 45 | 101 | 61.5 |
| control 7 | 188.5 | 101 | 62 | 71.5 | 169 | 152 | 51 |
| control 8 | 96.5 | 37 | 67 | 56 | 13 | 126 | 21 |
| Blank1 | 91 | 18.5 | 5 | 26.5 | 24 | 76 | 0 |
| Blank2 | 88 | 47 | 21 | 16.5 | 24.5 | 97.5 | 42.5 |

Table 2 shows scores of serum antibodies directed at HCV and KSHV antigens. The experiment was conducted simultaneously with beads coated with HCV and KSHV antigens mixed together. The score is calculated using the following equation: fluorescence unit/average fluorescence unit of the controls. Mix 1=HCV1+FH3832, Mix 2=HCV2+FH3834, Mix 3=HCV3+FI2129, Mix 4=HCV4+FF4913, Mix 5=HCV5+FG7779, Mix 6=HCV6+KSHV6. #: HCV2 is a negative sample according to BBI Diagnostics from which we purchased the samples.

TABLE 2

| | HCV | HCV | HCV | HCV | KSHV | KSHV | KSHV |
|---|---|---|---|---|---|---|---|
| | core | NS3 | NS4 | NS5 | K8.1 | ORF65 | ORF73 |
| HCV1 | 0.6 | 21.9 | 11.1 | 8.5 | 1.1 | 1.1 | 0.7 |
| HCV2^{#} | 0.4 | 1.5 | 1.4 | 1.3 | 0.9 | 0.8 | 0.3 |
| HCV3 | 78.0 | 3.2 | 2.9 | 2.8 | 0.5 | 2.4 | 2.1 |
| HCV4 | 122.1 | 7.9 | 12.5 | 4.4 | 1.3 | 2.6 | 2.4 |
| HCV5 | 28.5 | 12.2 | 2.4 | 2.7 | 0.5 | 4.5 | 0.7 |
| HCV6 | 2.2 | 79.8 | 4.8 | 3.3 | 1.6 | 1.6 | 1.2 |
| HCV7 | 15.3 | 5.0 | 3.3 | 2.2 | 0.9 | 1.3 | 1.2 |
| HCV8 | 8.7 | 38.8 | 1.0 | 4.6 | 0.8 | 0.9 | 0.5 |
| HCV9 | 1.0 | 81.3 | 1.8 | 0.8 | 0.3 | 3.4 | 0.9 |
| HCV10 | 25.5 | 46.7 | 6.7 | 5.7 | 4.0 | 1.2 | 1.1 |
| FH3832 | 2.0 | 0.9 | 1.5 | 2.7 | 29.0 | 26.3 | 94.8 |
| FH3834 | 1.8 | 0.9 | 2.0 | 1.7 | 13.4 | 22.7 | 85.8 |
| FI2129 | 2.0 | 0.9 | 1.9 | 2.1 | 46.1 | 17.3 | 72.2 |
| FF4913 | 3.4 | 1.1 | 2.8 | 2.4 | 27.6 | 46.4 | 163.4 |
| FG7779 | 2.0 | 0.9 | 2.7 | 1.6 | 15.7 | 14.6 | 96.0 |
| KSHV6 | 2.8 | 2.0 | 5.0 | 3.3 | 44.8 | 137.9 | 50.8 |
| Mix1 | 1.8 | 15.2 | 6.3 | 7.2 | 26.3 | 25.6 | 68.7 |
| Mix1 | 2.0 | 19.3 | 8.6 | 8.5 | 30.5 | 25.0 | 97.9 |
| Mix2 | 2.0 | 2.0 | 2.2 | 1.7 | 8.8 | 18.0 | 65.0 |
| Mix2 | 1.8 | 1.6 | 2.6 | 2.6 | 11.0 | 19.9 | 78.1 |
| Mix3 | 70.1 | 2.8 | 2.7 | 3.5 | 49.5 | 20.7 | 73.5 |
| Mix3 | 67.1 | 3.4 | 2.8 | 4.1 | 52.7 | 21.5 | 75.0 |
| Mix4 | 100.2 | 5.8 | 7.1 | 3.7 | 22.6 | 45.6 | 144.2 |
| Mix4 | 87.5 | 5.3 | 6.3 | 2.6 | 21.0 | 41.0 | 121.5 |
| Mix5 | 21.5 | 6.9 | 2.4 | 1.5 | 15.1 | 16.5 | 62.6 |
| Mix5 | 18.8 | 6.9 | 2.2 | 1.9 | 11.9 | 14.3 | 50.1 |
| Mix6 | 3.7 | 56.0 | 6.5 | 5.3 | 41.1 | 143.1 | 49.9 |
| Mix6 | 3.9 | 66.8 | 7.0 | 4.5 | 44.2 | 154.9 | 49.6 |
| control 1 | 0.6 | 0.4 | 1.4 | 0.8 | 0.3 | 1.1 | 1.1 |
| control 2 | 1.4 | 2.6 | 1.4 | 1.7 | 2.7 | 1.4 | 2.6 |
| control 3 | 1.4 | 1.5 | 1.6 | 0.9 | 1.3 | 0.7 | 1.1 |
| control 4 | 1.1 | 1.0 | 0.5 | 0.6 | 1.4 | 0.9 | 0.3 |
| control 5 | 0.9 | 0.1 | 0.4 | 0.6 | 0.2 | 0.8 | 0.0 |
| control 6 | 0.7 | 0.8 | 0.3 | 0.7 | 0.4 | 0.8 | 1.3 |
| control 7 | 1.3 | 1.1 | 1.2 | 1.4 | 1.6 | 1.2 | 1.1 |
| control 8 | 0.7 | 0.4 | 1.3 | 1.1 | 0.1 | 1.0 | 0.5 |

The results indicate that the detection method of the present invention is very sensitive for detecting the anti-viral antibodies. There is little cross-reactivity between antibodies directed at HCV and those against KSHV. Comparing results of Mix 1-6 to their corresponding single assay (e.g., Mix 1 to HCV1 and FH3832), the scores are quite reproducible, despite a potential interference between the two serum samples when mixed together.

### Example 3

Comparison of Results Generated by Renovar Assay and Ortho RIBA 3.0

Table 3

Recombinant Immunoblot assays (RIBA) have been developed by Ortho whereby nitrocellulose strips are coated with discrete bands of E. coli and yeast produced antigens (5,7-11). Detection of anti-KSHV antibodies by ELISA is based on a cocktail of immunodominant peptides from non-homologous regions of the respective HHV8 proteins. These peptides include specific KSHV antigens K8.1, orf73/LNA1, and orf65 (13-15). The HCV serum samples purchased from BBI Diagnostics were tested by Ortho RIBA 3.0 before their shipment to Renovar. The comparison of the test results generated from the Renovar assay and Ortho RIBA 3.0 is illustrated in Table 3. The correlation of the two methods is very good for antibodies directed at HCV core antigen, and is good for antibodies directed at the other 3 antigens. A perfect correlation is not expected, because the source of the antigens used are different, and the RIBA test itself is a semi-quantitative method.

TABLE 3

| | Core | Core | NS3 | NS3 | NS4 | NS4 | NS5 | NS5 |
|---|---|---|---|---|---|---|---|---|
| Sample | Renovar | RIBA | Renovar | RIBA | Renovar | RIBA | Renovar | RIBA |
| HCV #1 | 0.6 | — | 21.9 | 2+ | 11.1 | 2+ | 8.5 | 2+ |
| HCV #2 | 0.4 | — | 1.5 | — | 1.4 | — | 1.3 | — |
| HCV #3 | 78.0 | 4+ | 3.2 | — | 2.9 | — | 2.8 | 1+ |
| HCV #4 | 122.1 | 4+ | 7.9 | 2+ | 12.5 | — | 4.4 | — |
| HCV #5 | 28.5 | 2+ | 12.2 | ± | 2.4 | — | 2.7 | — |
| HCV #6 | 2.2 | — | 79.8 | 3+ | 4.8 | — | 3.3 | — |
| HCV #7 | 15.3 | 2+ | 5.0 | 1+ | 3.3 | — | 2.2 | — |
| HCV #8 | 8.7 | ± | 38.8 | 2+ | 1.0 | ± | 4.6 | — |
| HCV #9 | 1.0 | — | 81.3 | 3+ | 1.8 | 2+ | 0.8 | — |
| HCV #10 | 25.5 | 2+ | 46.7 | 1+ | 6.7 | ± | 5.7 | — |

### REFERENCES

1. Botte C, Janot C. Epidemiology of HCV infection in the general population and in blood transfusion. Nephrol Dial Transpl 1996; 11:19-21
2. Huang L, Koziel M J. Immunology of hepatitis C virus infection. Curr Opin Gastroen 2000; 6:558-564
3. Samuel M C, Doherty P M, Bulterys M, et al. Association between heroin use, needle sharing and tattoos received in prison with hepatitis B and C positivity among street-recruited injecting drug users in New Mexico, USA. Epidemiol Infect 2001; 127:475-484
4. Kempf W, Adams V. Viruses in the pathogenesis of Kaposi's sarcoma—A review Biochem Mol Med 1996; 58:1-12
5. Ensoli B, Sirianni M C. Kaposi's sarcoma pathogenesis: A link between immunology and tumor biology. Crit Rev Oncogenesis 1998; 9:107-124
6. Cesarman E, Knowles D M. The role of Kaposi's sarcoma-associated herpesvirus (KSHV/HHV-8) in lymphoproliferative diseases. Semin Cancer Biol 1999; 9:165-174
7. Jenner R G, Boshoff C. The molecular pathology of Kaposi's sarcoma-associated herpesvirus. BBA-Rev Cancer 2002; 1602:1-22
8. Laperche S, Courouce A M. Development of biological diagnosis of hepatitis C virus infection.
   Transfus Clin Biol 1997; 4:291-298
9. Pawlotsky J M, Lonjon I, Hezode C, et al. What strategy should be used for diagnosis of hepatitis C virus infection in clinical laboratories? Hepatology 1998; 27:1700-1702
10. Martin P, Fabrizi F, Dixit V, et al. Automated RIBA hepatitis C virus (HCV) strip immunoblot assay for reproducible HCV diagnosis. J Clin Microbiol 1998; 36:387-390
11. Ferrer F, Candela M J, Garcia C, et al. A comparative study of two third-generation anti-hepatitis C virus ELISAs. Haematologica 1997; 82:690-691
12. Fabrizi F, Martin P, Dixit V, et al. Automated RIBA (™) HCV strip immunoblot assay: A novel tool for the diagnosis of hepatitis C virus infection in hemodialysis patients. Am J Nephrol 2001; 21:104-111
13. Edelman D C, Ketema F, Saville R D, et al. Specifics on the refinement and application of two serological assays for the detection of antibodies to HHV-8. J Clin Virol 2000; 16:225-237
14. Rabkin C S, Schulz T F, Whitby D, et al. Interassay correlation of human herpesvirus 8 serologic tests. J Infect Dis 1998; 178:304-309
15. Chatlynne L G, Lapps W, Handy M, et al. Detection and titration of human herpesvirus-8-specific antibodies in sera from blood donors, acquired immunodeficiency syndrome patients and Kaposi's sarcoma patients using a whole virus enzyme-linked immunosorbent assay. Blood 1998; 92:53-58

All-publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of diagnosing Kaposi's sarcoma herpesvirus (KSHV) infection, comprising:
a) providing;
i) a sample from a subject, wherein said subject is suspected of having KSHV infection; and
ii) reagents for particle-based flow cytometric detection of at least one antibody from the group comprising antibodies to KSHV K8.1, orf73, and orf65 antigens; and
b) detecting the presence of said antibody in said sample using said reagents.

2. A kit, comprising:
a) reagents for particle-based flow cytometric detection of at least one antibody from the group comprising antibodies to KSHV K8.1, orf73, and orf65 antigens;
b) instructions for using said reagents for detecting the presence of at least one of said antibodies; and
c) instructions for using said detection of at least one of said antibodies in said sample for diagnosing KSHV infection.

3. A method of diagnosing hepatitis C virus (HCV) infection, comprising:
a) providing;
i) a sample from a subject, wherein said subject is suspected of having HCV infection; and
ii) reagents for particle-based flow cytometric detection of at least three or more antibodies from the group comprising antibodies to HCV core, NS3, NS4, and NS5 antigens; and
b) detecting the presence of said antibodies in said sample using said reagents.
